(19) United States
(12) Patent Application Publication
(10) Pub. No.: US 2024/0335119 A1
(43) Pub. Date: Oct. 10, 2024

(54) SYSTEM AND METHOD FOR USING MULTISPECTRAL IMAGING AND DEEP LEARNING TO DETECT GASTROINTESTINAL PATHOLOGIES BY CAPTURING IMAGES OF A HUMAN TONGUE

(71) Applicant: JUBAAN LTD, Yokne'am Illit (IL)

(72) Inventor: Asaf GOLAN, Zichron Yaakov (IL)

(21) Appl. No.: 18/293,476

(22) PCT Filed: Jul. 26, 2022
(86) PCT No.: PCT/IL2022/050803

§ 371 Date: Jan. 30, 2024

Related U.S. Application Data
(60) Provisional application No. 63/228,824, filed on Aug. 3, 2021.

(51) Int. Cl. A61B 5/00

(52) U.S. Cl. CPC A61B 5/0088; A61B 5/0075; A61B 5/0077; A61B 5/42; A61B 5/4854; A61B 5/7267; A61B 5/7282; A61B 2562/0233; A61B 2576/02

(57) ABSTRACT

Systems and methods configured to classify a multispectral image as being associated with one or more gastrointestinal disorders, based, at least in part, on one or more ranges of wavelengths within output data, wherein the output data includes a product of a specific combination of operations corresponding to one or more gastrointestinal disorders onto a cube associated with the multispectral image, the cube including a plurality of superpixels wherein each superpixel is associated with spatial coordinates on a tongue of a subject, and wherein the plurality of superpixels are received from one or more image capturing devices.

### TECHNICAL FIELD

The present disclosure relates generally to diagnosis of gastrointestinal disorders using multispectral imaging of a human tongue.

### BACKGROUND

Human Tongue analysis is a common diagnostic tool in traditional Chinese medicine. Observation of a tongue of a subject enables practitioners to diagnose symptoms and/or pathologies of the subject. Some of the characteristics of the tongue which are observed by the practitioners are shape, color, texture, geometry, and morphology. By observing such characteristics, practitioners are able to detect pathologies of the subject in a non-invasive manner.

The foregoing examples of the related art and limitations related therewith are intended to be illustrative and not exclusive. Other limitations of the related art will become apparent to those of skill in the art upon a reading of the specification and a study of the figures.

### SUMMARY

According to some embodiments there is provided a system for detection of disorders based on one or more multispectral images of a tongue of a subject. According to some embodiments, the system may be configured to capture one or more multispectral images of a tongue of a subject simultaneously, thereby reducing the time needed to obtain image data of the tongue. According to some embodiments, the system and method may be configured to merge two or more images of the tongue of the subject into a single multispectral image, in the form of a cube. According to some embodiments, the systems and methods may be configured to classify the single multispectral image (or cube) as being associated with one or more gastrointestinal (GI) disorders based, at least in part, on the depicted ranges of wavelengths depicted in output data resulting from one or more combinations of operations applied to the cube.

According to some embodiments, the systems and methods may be configured to generate, using a machine learning algorithm, a specific combination of operations for conversion of the cube, and wherein the combination of operations corresponds to one or more GI disorders. According to some embodiments, the systems and methods may be configured to classify the single multispectral image as being associated with a GI disorder based, at least in part, on the depicted ranges of wavelengths depicted in the output data.

According to some embodiments there is provided a system for detecting gastrointestinal disorders utilizing one or more multispectral images of a tongue of a subject, including: at least one hardware processor in communication with the at least one image capturing device configured to capture at least one multispectral image of a tongue of the subject in real time, and a non-transitory computer-readable storage medium having stored thereon program code, the program code executable by the at least one hardware processor to: receive the at least one multispectral image obtained by the at least one image capturing device, wherein the at least one multispectral image includes at least one superpixel associated with spatial coordinates on the tongue of the subject, each pixel of the at least one superpixel depicting a specified range of wavelengths of light, generate a cube, based on the superpixel, of the at least one multispectral images, the cube including at least: a first and second dimensions associated with spatial coordinates on the tongue of the subject, and a third dimension associated with ranges of wavelengths of light corresponding to the spatial coordinates on the tongue of the subject, generate, using a machine learning algorithm, a specific combination of operations for conversion of the cube, and wherein the combination of operations corresponds to one or more gastrointestinal disorders.

According to some embodiments there is provided a system for detecting gastrointestinal disorders utilizing one or more multispectral images of a tongue of a subject, including: at least one hardware processor in communication with the at least one image capturing device configured to capture at least one multispectral image of a tongue of the subject in real time, and a non-transitory computer-readable storage medium having stored thereon program code, the program code executable by the at least one hardware processor to: receive the at least one multispectral image obtained by the at least one image capturing device, wherein the at least one multispectral image includes at least one superpixel associated with spatial coordinates on the tongue of the subject, each pixel of the at least one superpixel depicting a specified range of wavelengths of light, generate a cube, based on the at least one superpixel of the at least one multispectral image, the cube including at least: a first and second dimensions associated with spatial coordinates on the tongue of the subject, and a third dimension associated with ranges of wavelengths of light corresponding to the spatial coordinates on the tongue of the subject, convert the cube, into output data, using a specific combination of operations corresponding to one or more gastrointestinal disorders, and classify the output data as being associated with one or more gastrointestinal disorders, based, at least in part, on one or more ranges of wavelengths depicted within the output data.

According to some embodiments, the at least one superpixels is in the form of a 3 by 3 or 4 by 4 or 5 by 5 or 6 by 6 matrix.

According to some embodiments, the combination of operations is configured to emphasize at least one mathematical relationship between two or more planes of the cube associated with one or more gastrointestinal disorders.

According to some embodiments, the cube includes a plurality of planes along the third dimension, each plane corresponding to a wavelength or range of wavelengths.

According to some embodiments, the combination of operations is configured to provide a specific weight to different planes of the cube.

According to some embodiments, the combination of operations includes at least one specific operation that is applied to only a portion of the plurality of planes.

According to some embodiments, the combination of operations includes at least one linear operation.

According to some embodiments, the combination of operations includes at least one non-linear operation.

According to some embodiments, the combination of operations is configured to reduce the number of total planes of the converted matrix in relation to the cube.

According to some embodiments, the machine learning algorithm is trained using a training set including: a plurality of cubes corresponding to a plurality of tongues of a plurality of subjects, and a plurality of labels associated with the plurality of cubes, each label indicating at least one medical disorder associated with the corresponding plurality of subjects.

According to some embodiments, the at least one hardware processor is in communication with at least one image capturing device, wherein the at least one image capturing device is configured to capture at least one multispectral image of a tongue of a subject in real time.

According to some embodiments, the specific combination of operations is generated using a machine learning algorithm.

According to some embodiments, the output data includes a single multispectral image.

According to some embodiments, the output data includes one or more submatrices, one or more images, a series of images one or more scalar signals, or any combination thereof.

According to some embodiments, the cube includes a plurality of planes along the third dimension, each plane corresponding to a wavelength or range of wavelengths.

According to some embodiments, the program code is further executable to classify the single multispectral image as being associated with one or more gastrointestinal disorders, based, at least in part, on a machine learning algorithm configured to receive the converted cube and output one or more gastrointestinal disorders corresponding to any one or more of the values of one or more pixels, proportions or relative weights of the planes, amplitudes of specific wavelengths or ranges of wavelengths and intensities of specific wavelengths or ranges of wavelengths, of the converted cube.

According to some embodiments, the at least one image capturing device includes at least one camera.

According to some embodiments, the at least one image capturing device includes at least one lens.

According to some embodiments, the at least one hardware processor is in communication with at least one image capturing device, wherein the at least one image capturing device is configured to capture at least one multispectral image of a tongue of a subject in real time.

According to some embodiments, the at least one image capturing device includes at least one camera configured to capture at least one multispectral image of a tongue of a subject in real time.

According to some embodiments, the at least one image capturing device includes at least two cameras and wherein the at least two cameras are positioned in an optical path of a beamsplitter such that each of the at least two cameras obtains a separate spectrum of light reflected from the tongue.

According to some embodiments, the beamsplitter is positioned such that at least one angles of incidence of the optical path is between about 30 and 65 degrees.

According to some embodiments, the at least one image capturing device include at least two sensors, wherein each sensor includes a plurality of lenses each configured to capture a wavelength or range of wavelengths.

According to some embodiments, the program code is further executable to pre-process the cube, wherein pre-processing includes segmentation, accounting for motion blur, distortion, and/or data replication caused by motion of the tongue during the capturing of the image.

According to some embodiments, the program code is further executable to convert the cube to an RGB image prior to and/or during a pre-processing stage.

According to some embodiments, the program code is further executable to merge two or more images obtained from the at least one image capturing device.

According to some embodiments, the multispectral images include between about 30 and 60 wavelengths or ranges of wavelengths.

According to some embodiments, the at least one hardware processor is in communication with at least two image capturing devices and wherein the ranges of wavelengths obtained by the at least two image capturing devices are different.

According to some embodiments, the at least one hardware processor is in communication with at least two image capturing devices and wherein the ranges of wavelengths obtained by the at least two image capturing devices at least partially overlap.

According to some embodiments, the wavelengths obtained by the at least one image capturing device range within any one or more of visible light, ultraviolet light, near infrared light, and infrared light wavelengths.

According to some embodiments, at least one of the ranges of wavelengths range within 470 and 900 nm.

According to some embodiments, the at least one multispectral image includes a video segment.

According to some embodiments, the at least one image capturing device is configured to capture the video segment within no more than about 100 msec According to some embodiments, the at least one image capturing device is configured to capture the at least one image at a depth of field of about 100 mm.

According to some embodiments, the at least one image capturing device includes a field of view of about 150 mm by 100 mm.

According to some embodiments, a maximal exposure time of the at least one image captured by the at least one image capturing device is about 100 msec.

According to some embodiments, the multispectral image includes over about 25 active bands.

According to some embodiments, the at least one hardware processor is in communication with at least two image capturing devices and wherein the program code is further executable to operate the at least two image capturing devices simultaneously.

According to some embodiments there is provided a method for detecting gastrointestinal disorders utilizing one or more multispectral images of a tongue of a subject, the method including: obtaining a plurality of multispectral images of a tongue of the subject wherein each image includes at least one superpixel, depicting a specified range of wavelengths of light reflected from the tongue of the subject, merging the plurality of multispectral images, thereby forming a cube including at least: a first and second dimensions associated with spatial coordinates on the tongue of the subject, and a third dimension associated with ranges of wavelengths of light corresponding to the spatial coordinates on the tongue of the subject, and generating, using a machine learning algorithm, a specific combination of operations for conversion of the cube, and wherein the combination of operations corresponds to one or more gastrointestinal disorders.

According to some embodiments there is provided a method for detecting gastrointestinal disorders utilizing one or more multispectral images of a tongue of a subject, the method including: obtaining a plurality of multispectral images of a tongue of the subject wherein each image includes at least one superpixels, depicting a specified range of wavelengths of light reflected from the tongue of the subject, merging the plurality of multispectral images, thereby forming a cube including at least: a first and second dimensions associated with spatial coordinates on the tongue of the subject, and a third dimension associated with ranges of wavelengths of light corresponding to the spatial coordinates on the tongue of the subject, converting the cube, into output data, based, at least in part, on a specific combination of operations corresponding to one or more gastrointestinal disorders, and classifying the output data as being associated with one or more gastrointestinal disorders, based, at least in part, on the one or more ranges of wavelengths depicted by the output data.

According to some embodiments, the specific combination of operations is generated using a machine learning algorithm.

According to some embodiments, the output data includes a single multispectral image.

According to some embodiments, the output data includes one or more submatrices, one or more images, a series of images one or more scalar signals, or any combination thereof.

According to some embodiments, the cube includes a plurality of planes along the third dimension, each plane corresponding to a wavelength or range of wavelengths.

According to some embodiments, the method further includes classifying the single multispectral image as being associated with one or more gastrointestinal disorders, based, at least in part, on a machine learning algorithm configured to receive the converted cube and output one or more gastrointestinal disorders corresponding to any one or more of the values of one or more pixels or subset of pixels, proportions of the planes, amplitudes of specific wavelengths or ranges of wavelengths, and intensities of specific wavelengths or ranges of wavelengths, of the converted cube.

According to some embodiments, the method further includes pre-processing the cube, wherein pre-processing includes segmentation, accounting for motion blur, distortion, and/or data replication caused by motion of the tongue during the capturing of the image.

According to some embodiments, the method further includes converting the cube to an RGB image prior to and/or during a pre-processing stage.

According to some embodiments, the multispectral images include between about 30 and 60 wavelengths or ranges of wavelengths.

According to some embodiments, the ranges of wavelengths are different.

According to some embodiments, the ranges of wavelengths at least partially overlap.

According to some embodiments, the wavelengths range within any one or more of visible light, ultraviolet light, near infrared light, and infrared light wavelengths.

According to some embodiments, at least one of the ranges of wavelengths range within 470 and 900 nm.

According to some embodiments, the at least one multispectral image includes a video segment.

According to some embodiments, the video segment is captured within no more than about 100 msec According to some embodiments, the multispectral image includes over about 25 active bands.

According to some embodiments, the multispectral image includes one or more bands having a width of less than about 20 nm.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 shows a schematic simplified illustration of a system for detection of gastrointestinal disorders, in accordance with some embodiments of the present invention;

FIG. 2A, FIG. 2B, and FIG. 2C show simplified illustrations of exemplary superpixels, in accordance with some embodiments of the present invention;

FIG. 2D shows a simplified illustration of exemplary pixels collected from a lens, in accordance with some embodiments of the present invention;

FIG. 3 shows a simplified illustration of an exemplary array associated with a spatial coordinate, in accordance with some embodiments of the present invention;

FIG. 4 shows a simplified illustration of an exemplary cube, in accordance with some embodiments of the present invention;

FIG. 5 shows a simplified illustration of a tongue with exemplary segmentations, in accordance with some embodiments of the present invention;

FIG. 6 shows a flowchart of functional steps in a process for detection of gastrointestinal disorders, in accordance with some embodiments of the present invention; and

FIG. 7 shows a flowchart of functional steps in a process for detection of gastrointestinal disorders, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

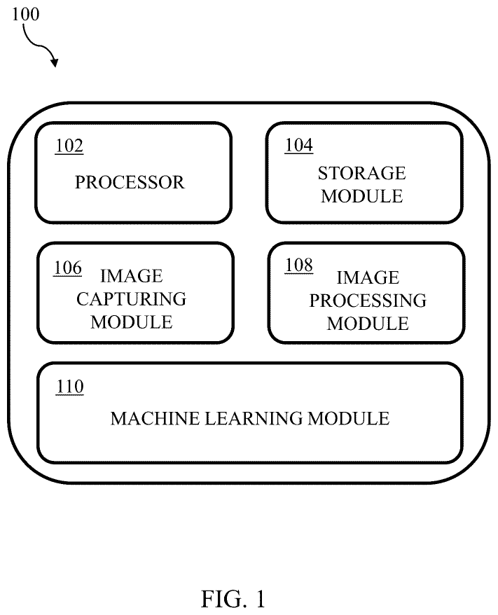

Reference is made to FIG. 1, which shows a schematic simplified illustration of a system for detection disorders, in accordance with some embodiments of the present invention.

According to some embodiments, the system 100 may be configured to detect gastrointestinal (GI) disorders based, at least in part, on one or more multispectral images of a tongue of a subject. According to some embodiments, the gastrointestinal disorders may include any one or more of gastrointestinal disorders, lower gastrointestinal disorders, and/or upper gastrointestinal disorders. According to some embodiments, the system 100 may include at least one of a hardware processor 102, a storage module 104, an image capturing module 106, an image processing module 108, and a machine learning module 110. According to some embodiments, each possibility is a separate embodiment. According to some embodiments, the machine learning module 110 may be configured to receive the one or more multispectral images of a tongue of a subject and output one or more gastrointestinal disorders associated with the tongue of the subject.

According to some embodiments, the processor 102 may be in communication with at least one of the storage module 104, the image capturing module 106, the image processing module 108, and the machine learning model 110. According to some embodiments, the processor 102 may be configured to control operations of any one or more of the storage module 104, the image capturing module 106, the image processing module 108, and the machine learning model 110. According to some embodiments, each possibility is a separate embodiment.

According to some embodiments, the storage module 104 may include a non-transitory computer-readable storage medium. In some embodiments, the storage module 104 may include one or more program code and/or sets of instructions for detection of disorders. According to some embodiments, the program code may be configured to instruct the use of and/or command the operation of at least one of the processor 102, the image capturing module 106, the image processing module 108, and the machine learning module 110. According to some embodiments, each possibility is a separate embodiment.

According to some embodiments, the storage module 104 may include one or more algorithms configured to detect disorders, based on, at least in part, one or more images of a tongue of the subject, for example, such as, using method 200 or method 300, and as described in greater detail elsewhere herein.

According to some embodiments, the image capturing module 106 may be configured to obtain at least one image of a tongue of the subject. According to some embodiments, the image capturing module 106 may be configured to obtain at least one multispectral image of a tongue of the subject. According to some embodiments, the image capturing module 106 may be configured to obtain at least one Hyperspectral image of a tongue of the subject. According to some embodiments, the image capturing module 106 may include one or more image capturing devices. According to some embodiments, the image capturing module 106 may include two or more image capturing devices. According to some embodiments, the image capturing devices may include any one or more of a camera, lens, and sensor, or any combination thereof.

According to some embodiments, the system 100 and/or the image capturing module 106 may be configured to illuminate the tongue of the subject during the capturing of the images. According to some embodiments, the processor 102 may be configured to control the illumination modes of the illumination of the tongue of the subject during the capturing of the images. According to some embodiments, the processor 102 may be configured to control one or more of the intensity, and wavelengths, of light projected, or illuminated, onto the tongue of the subject.

According to some embodiments, the image capturing module 106 and/or the image capturing device may be configured to capture one or more images of a tongue of a subject in real time. According to some embodiments, the image capturing module 106 may be configured to send data associated with the one or more images of the tongue to any one or more of the processor 102, the memory module 104, the image processing module 108, and the machine learning module 110. According to some embodiments, the image capturing module 106 may be configured to send data associated with the one or more images of the tongue in real time.

According to some embodiments, the image may be a portion of a video segment.

According to some embodiments, the one or more image capturing devices may be configured to capture one or more video segments. According to some embodiments, the image may include a plurality of frames. According to some embodiments, the video segment may be captured within no more than 100 msec. According to some embodiments, the video segment may be captured within no more than 70 msec.

According to some embodiments, the video segment may be captured within no more than 40 msec. According to some embodiments, the video segment may be captured within no more than 20 msec.

According to some embodiments, the processor 102 may be configured to command the image capturing module 106 to obtain and/or capture the one or more images. According to some embodiments, the processor 102 may be configured to command the image capturing module 106 to obtain and/or capture the one or more videos. According to some embodiments, the image capturing module 106 may include at least one image capturing device and/or at least one coupler configured to communicate between system 100 and one or more image capturing device. For example, according to some embodiments, the image capturing module 106 may include at least one camera.

For example, according to some embodiments, the image capturing module 106 may include one or more sensors, such as CMOS sensors. According to some embodiments, the coupler may include at least one cable or wireless connection through which the processor 102 may obtain the one or more images from the one or more image capturing device.

According to some embodiments, the one or more image capturing devices may be configured to capture at least one image, such as a multispectral image, of the tongue of the subject. According to some embodiments, the one or more image capturing devices may be configured to capture at least one image of the tongue of the subject in real time. According to some embodiments, the processor 102 may be configured to operate the one or more image capturing devices simultaneously.

According to some embodiments, the image capturing module 106 may include two or more image capturing devices. According to some embodiments, the two or more image capturing devices may be each configured to capture at least one image, such as a multispectral image, of the tongue of the subject. According to some embodiments, the two or more image capturing devices may be each configured to capture at least one image of the tongue of the subject in real time. According to some embodiments, the processor 102 may be configured to operate two or more image capturing devices simultaneously.

Advantageously, capturing two images simultaneously enables capturing images of the tongue in real time and in multiple wavelengths since each camera may be active in different wavelength band of frequencies.

According to some embodiments, the system 100 may include a frame which may include a forehead rest configured to position the face of the subject. According to some embodiments, two or more image capturing devices may be positioned at different angles in relation to the frame, such that the two or more image capturing devices may capture images from two or more angles in relation to the tongue of the subject. According to some embodiments, the position of the one or more image capturing devices may be moveable in relation to any one or more of the frame, the forehead rest, and/or the tongue of the subject. According to some embodiments, the position of the one or more image capturing devices may be fixed in relation to any one or more of the frame, the forehead rest, and/or the tongue of the subject.

According to some embodiments, the at least one image capturing device may include one or more cameras. According to some embodiments, the system 100 may include a beamsplitter. According to some embodiments, the beamsplitter may be configured to split beams of light reflected from the tongue of the subject towards the two or more image capturing devices. According to some embodiments, the beamsplitter may be positioned in an optical path of two or more image capturing devices such that the two or more image capturing devices obtains a separate spectrum of light reflected from the tongue.

According to some embodiments, the beamsplitter may be positioned such that at least one angle of incidence of the optical path is between about 20 to 70 degrees. According to some embodiments, the beamsplitter may be positioned such that at least one angles of incidence of the optical path is between about 35 to 55 degrees. According to some embodiments, the beamsplitter may be positioned such that at least one angles of incidence of the optical path is between about 20 to 50 degrees. According to some embodiments, the beamsplitter may be positioned such that at least one angles of incidence of the optical path is between about 38.6 and 52.3 degrees.

According to some embodiments, the at least one image capturing device may include one or more sensors. According to some embodiments, the at least one image capturing device may include two or more sensors. According to some embodiments, the sensors may include a plurality of lenses each configured to capture a wavelength or range of wavelengths. According to some embodiments, the sensors may each include a plurality of lenses each configured to capture a wavelength or range of wavelengths. According to some embodiments, the plurality of lenses may include at least 16 lenses. According to some embodiments, the plurality of sensors may include between about 10 and 36 lenses.

According to some embodiments, the system 100 may include a prism. According to some embodiments, the prism may be positioned in an optical path of the lenses such that light reflected from the tongue of the subject is separated into the plurality of lenses.

According to some embodiments, the image capturing devices may be configured to capture any one or more of hyperspectral images and/or multispectral images. According to some embodiments, the images may include between about 30 and 60 wavelengths or ranges of wavelengths. According to some embodiments, at least one of the images may include between about 5 and 500 bands. According to some embodiments, at least one of the images may include between about 15 and 100 bands. According to some embodiments, at least one of the images may include between about 20 and 50 bands. According to some embodiments, at least one of the images may include over about 25 active bands. According to some embodiments, the band width of at least a portion of the bands may be under about 30 nm. According to some embodiments, the band width of at least a portion of the bands may be under about 20 nm.

According to some embodiments, the one or more image capturing devices and/or the system 100 may be configured such that the ranges of wavelengths obtained by different image capturing device are different. According to some embodiments, the image capturing devices and/or the system 100 may be configured such that the wavelengths obtained by two image capturing devices are different. According to some embodiments, the beamsplitter and/or prism may be configured to split the different wavelengths to two or more image capturing devices. According to some embodiments, the beamsplitter and/or prism may be configured to split the different ranges of wavelengths to two or more image capturing devices.

According to some embodiments, the ranges of wavelengths and/or wavelengths obtained by at least one of two or more image capturing devices may at least partially overlap. According to some embodiments, the wavelengths ranges and/or the wavelengths may include any one or more of visible light, ultraviolet light, near infrared light, and infrared light wavelengths. According to some embodiments, the ranges of wavelengths and/or wavelengths obtained by the at least one image capturing device may range between about 470 and 900 nm.

According to some embodiments, the at least one image capturing device may be configured to capture the images at a depth of field of about 100 mm. According to some embodiments, the at least one image capturing device may be configured to capture the images at a depth of field of between about 50 mm to 150 mm. According to some embodiments, the at least one image capturing device may include a field of view of about 150 mm by 100 mm.

According to some embodiments, the one or more image capturing devices may be configured to capture one or more images having a maximal exposure time configured to avoid motion blur of the image data. According to some embodiments, the maximal exposure time may be about 100 msec. According to some embodiments, the maximal exposure time may be under 100 msec. According to some embodiments, the maximal exposure time may be between about 0.5 msec and 90 msec.

According to some embodiments, the image capturing module 106 may include and/or be in communication with one or more sensors configured to capture signals associated with movement of a tongue of a subject. According to some embodiments, the one or more sensors may include a motion sensor. According to some embodiments, the system 100 may include one or more algorithms configured to receive data from the one or more sensors and identify and/or detect motion of the tongue of the subject. According to some embodiments, the system 100 may include one or more algorithms configured to identify/detect motion of the tongue of the subject based, at least in part, on the one or more captured images.

According to some embodiments, one or more of program stored onto the storage module 104 may be executable to capture the one or more images. According to some embodiments, the processor 102 may be configured to command the capture of the one or more images in real time while receiving image data from the image capturing module 106. According to some embodiments, the image capturing module 106 and/or system 100 may be configured to receive the one or more images from a plurality of different types of image capturing devices. According to some embodiments, and as described in greater detail elsewhere herein, the system 100 may be configured to normalize different images which may be captured by more than one type of image capturing device, using the image processing module 108.

According to some embodiments, the image processing module 108 may be configured to receive the one or more images captured and/or obtained by the image capturing module 106. According to some embodiments, the storage module 104 may include a cloud storage unit. According to some embodiments, the processor 102 may be in communication with a cloud storage unit. According to some embodiments, the image processing module 108 and/or the machine learning module 110 may be stored onto a cloud storage unit and/or the storage module 104. According to some embodiments, the storage module 104 may be configured to receive the one or more images by uploading the images onto the cloud storage unit and/or the storage module 104.

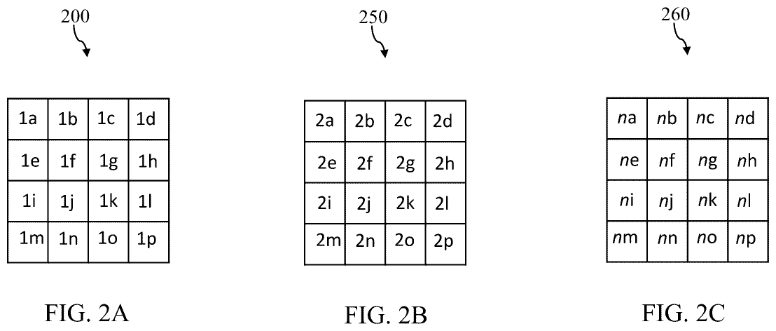

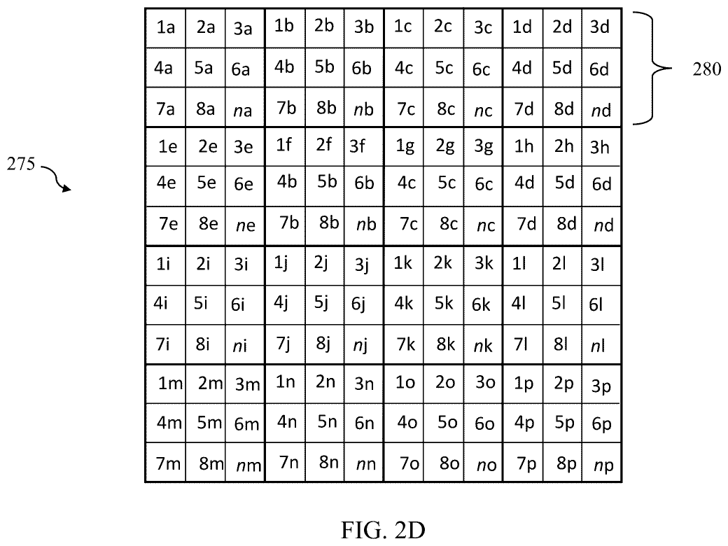

Reference is made to FIG. 2A, FIG. 2B, and FIG. 2C, which are simplified illustrations of exemplary superpixels, in accordance with some embodiments of the present invention.

According to some embodiments, the processing algorithm may be configured to receive the one or more images. According to some embodiments, the processing algorithm may be configured to receive the one or more images after the one or more images are applied to one or more pre-processing algorithm. According to some embodiments, the processing algorithm may be configured to receive the one or more images in the form of superpixels. According to some embodiments, the processing algorithm may be configured to transform the one or more images to the form of superpixels. According to some embodiments, each area of the tongue (or location on the tongue), or one or more spatial coordinates of the image, may include one or more superpixels. According to some embodiments, each image may be transformed into a form which includes a plurality of superpixels. According to some embodiments, each superpixel within an image may be associated with one or more spatial coordinates within the image and/or on the tongue of the subject. According to some embodiments, each superpixel of an image may be associated with a specific spatial coordinate of the image and/or on the tongue of the subject.

According to some embodiments, the one or more superpixels may include a plurality of pixels therein. According to some embodiments, each pixel within a superpixel may depict a specified range of wavelengths of light. According to some embodiments, the one or more pixels in each of the superpixels may be in the form of a matrix. According to some embodiments, the matrix may include a 4 by 4 structure, such as the superpixels 200, 250, and 260 as depicted in FIG. 2A, FIG. 2B, and FIG. 2C, respectively. According to some embodiments, the matrix may include a 5 by 5 structure.

According to some embodiments, the matrix may include a symmetrical or non-symmetrical structure such as, for example: 3 by 3, 4 by 4, 5 by 5, 6 by 3, 8 by 10, 2 by 5, or the like.

According to some embodiments, each pixel within a superpixel may depict a specified narrow band of wavelength of light. According to some embodiments, each pixel may include a value associated with an intensity of the light within the range of wavelengths. According to some embodiments, at least a portion of the pixels within a superpixel may depict a specified wavelength of light. According to some embodiments, each pixel within a superpixel may depict a different range of wavelengths of light than other pixels within the superpixel. According to some embodiments, at least a portion of the pixels within a superpixel may depict one or more different ranges of wavelengths of light than the other pixels within the superpixel. For example, the superpixels 200, 250, and 260 as depicted in FIG. 2A, FIG. 2B, and FIG. 2C, respectively, each include 16 pixels (labeled 1a-1p, 2a-2p, and na-np within each of the superpixels 200 and 250, respectively), wherein each of the pixels 1a-1p, 2a-2p, and na-np may include a range of wavelengths.

For example, according to some embodiments, the image may include n superpixels. According to some embodiments, each of the n superpixels may be associated with a coordinate or area on the tongue. According to some embodiments, each of the n superpixels may include a plurality of pixels, such as the pixels 1a-1p, 2a-2p, and na-np depicted in FIG. 2A, FIG. 2B, and FIG. 2C. According to some embodiments, each of the pixels may be associated with a band of wavelength of light. For example, the pixels 1a, 2a, and so forth, up to pixel na, may depict a same band of wavelength of light, such as, for example, 400 nm to 430 nm, 610 nm to 620 nm, and the like. Similarly, the pixels 1b, 2b, and so forth, up to pixel nb, may depict a same band of wavelength of light, and so on.

According to some embodiments, the one or more images captured and/or obtained by the image capturing module 106 may include two or more overlapping spatial coordinates of the tongue of the subject, or in other words, may depict a same spatial coordinate of the tongue of the subject. According to some embodiments, two or more images captured and/or obtained by the image capturing module 106 may include two or more overlapping spatial coordinates of the tongue of the subject, or in other words, may depict a same spatial coordinate of the tongue of the subject. According to some embodiments, the one or more images captured and/or obtained by the image capturing module 106 may include two or more superpixels associated with a same spatial coordinate of the tongue of the subject. According to some embodiments, two or more superpixels associated with a same spatial coordinates may include a same or different number of pixels therein.

According to some embodiments, two or more superpixels associated with a same spatial coordinates may include a same or different ranges of wavelengths associated with the pixels within the two or more superpixels. For example, the range of wavelengths depicted in pixel 1p of superpixel 200 may include one or more wavelengths which may be depicted in pixel 2a of superpixel 250. According to some embodiments, different superpixels (associated with a same spatial coordinates on the tongue of the subject) originating from the same or different images may include completely different ranges of wavelengths. According to some embodiments, different superpixels (associated with a same spatial coordinates on the tongue of the subject) originating from different images may include at least some different ranges of wavelengths. According to some embodiments, different superpixels (associated with a same spatial coordinates on the tongue of the subject) originating from the same or different images may include at least some same ranges of wavelengths. For example, the ranges of wavelengths depicted by pixels 1a-1p of superpixel 200 may include ranges of wavelengths that are not depicted by any one of pixels 2a-2p of superpixel 250. According to some embodiments, and as described in greater detail elsewhere herein, the image processing module 108 may be configured to generate an array of pixels from the one or more superpixels.

Reference is made to FIG. 2D, which is a simplified illustration of exemplary pixels collected from a lens, in accordance with some embodiments of the present invention.

According to some embodiments, and as described in greater detail elsewhere herein, the image capturing module may include one or more lenses. According to some embodiments, the image capturing module may include one or more sensors positioned such that light passing through the one or more lenses is captured by the one or more sensors. According to some embodiments, the one or more sensors may be configured to convert the light passing through the one or more lenses into pixels, which may be organized into a matrix, such as the matrix 275 depicted in FIG. 2D. According to some embodiments, and as described in greater detail elsewhere herein, the image processing module 108 may be configured to generate an array of pixels from the matrix captured using the one or more lenses. According to some embodiments, the one or more lenses may be used to capture an image including one or more pixels, which may be used to generate an array of pixels as described herein.

For example, according to some embodiments, the image may include a matrix, such as matrix 275, including a plurality of submatrices 280. According to some embodiments, the plurality of submatrices 280 may all (or most) depict pixels associated with the same coordinates or areas on the tongue. According to some embodiments, each of the submatrices 280 may be each associated with a specific band of wavelength of light. According to some embodiments, each of the plurality of submatrices 280 may include a plurality of pixels. According to some embodiments, each submatrix 280 may include n pixels. According to some embodiments, each of the n pixels of a submatrix 280 may be associated with a band of wavelength of light. For example, the pixels 1a, 2a, and so forth, up to pixel na, may depict a same band of wavelength of light, such as, for example, 540 nm to 555 nm, 800 nm to 840 nm, and the like. Similarly, the pixels 1b, 2b, and so forth, up to pixel nb, may depict a same band of wavelength of light, and so on.

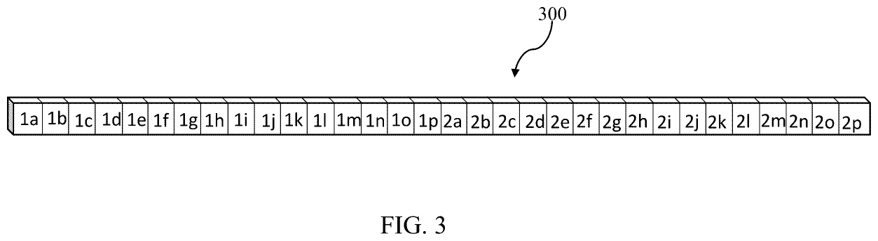

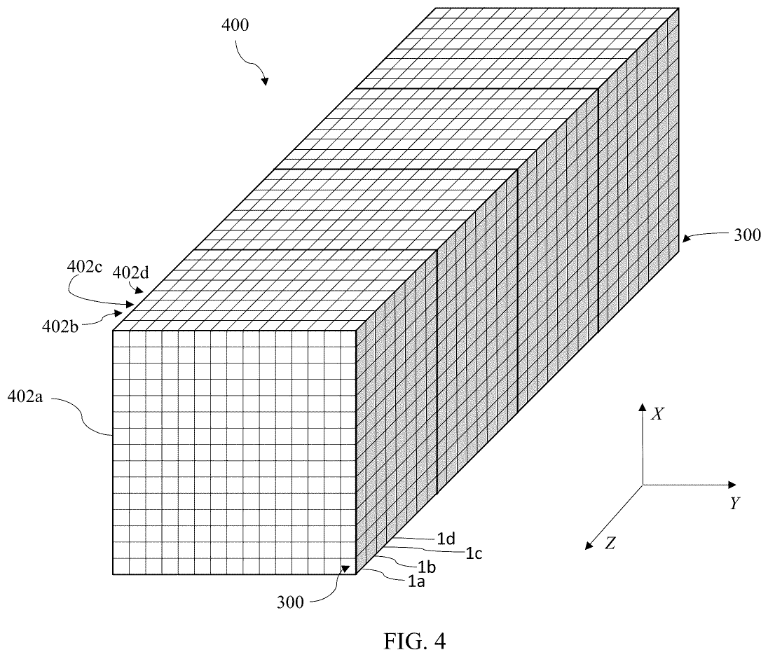

Reference is made to FIG. 3, which is a simplified illustration of an exemplary array associated with a spatial coordinate, in accordance with some embodiments of the present invention, and to FIG. 4, which is a simplified illustration of an exemplary cube, in accordance with some embodiments of the present invention.

According to some embodiments, the image processing module 108 may be configured to generate one or more array of pixels, such as array 300. According to some embodiments the image processing module 108 may be configured to generate one or more array of pixels wherein each array of pixels includes a plurality of pixels depicting different wavelengths or ranges of wavelengths associated with a spatial coordinate of the image and/or the tongue of the subject.

According to some embodiments, the image processing module 108 may be configured to generate an array of pixels from the one or more superpixels. According to some embodiments, the image processing module 108 may be configured to generate an array of pixels from the matrix including the pixels depicting the light transmitted through the one or more lenses. It is to be understood that the system may be configured to generate an array of pixels using any output data of an image capturing device which depicts a plurality of pixels, preferably wherein each pixel may depict a wavelength or range of wavelengths.

According to some embodiments, the image processing module 108 and/or the image processing algorithm may be configured to align the one or more pixels within the one or more arrays of pixels. According to some embodiments, the image processing module 108 and/or the image processing algorithm may be configured to merge two or more arrays generated using two or more superpixels. According to some embodiments, the image processing module 108 and/or the image processing algorithm may be configured to merge two or more arrays of two or more superpixels, wherein the two or more superpixels correspond to a same spatial coordinate on the tongue of the subject.

According to some embodiments, the image processing module 108 and/or the image processing algorithm may be configured to generate an array (or merged array) based, at least in part, on two or more superpixels of the one or more images or two or more images, wherein the two or more superpixels may be associated with a same spatial coordinate on the tongue of the subject. According to some embodiments, the merged array may include at least a portion of the pixels of the two or more superpixels, for example, as depicted by merged array 300 in FIG. 3, which includes all of the pixels 1a-1p and 2a-2p as depicted in the two superpixels 200 and 250. According to some embodiments, the image processing module 108 and/or the image processing algorithm may be configured to arrange the pixels within the merged array. According to some embodiments, the image processing module 108 and/or the image processing algorithm may be configured to arrange the pixels within the merged array for example, by wavelength and/or intensity. According to some embodiments, the image processing module 108 and/or the image processing algorithm may be configured to evaluate and/or compare the pixels within the merged array. According to some embodiments, the image processing module 108 and/or the image processing algorithm may be configured to remove at least one of two or more pixels which may be replicas of each other.

According to some embodiments, pixels which may be replicas of each other, or in other words, replica pixels, may include two or more pixels that both depict a same wavelength and/or a same range of wavelengths. According to some embodiments, for two or more pixels which may be replicas of each other (referred to herein as replica pixels), the image processing module 108 and/or the image processing algorithm may be configured to generate a new pixel for the merged array which would replace the two or more replica pixels, wherein the new pixel may depict the same wavelength and/or range of wavelengths of the two or more replica pixels.

According to some embodiments, the image processing module 108 and/or the image processing algorithm may be configured to calculate the intensity of the wavelength and/or range of wavelengths depicted by the new pixel based on the intensity of the wavelength and/or range of wavelengths depicted by at least one of the replica pixels. According to some embodiments, the image processing module 108 and/or the image processing algorithm may be configured to select the intensity of the wavelength and/or range of wavelengths depicted by the new pixel, from the intensities of the depicted wavelengths and/or ranges of wavelengths of the two or more replica pixels.

According to some embodiments, the image processing module 108 and/or the image processing algorithm may be configured to merge two or more images obtained and/or captured by the one or more image capturing devices 106. According to some embodiments, the merging of the two or more images may include merging a plurality of superpixels of two or more images, wherein each pair (or group) of merged superpixels may be associated with a same spatial coordinate on images and/or the tongue of the subject. According to some embodiments, the image processing module 108 and/or the image processing algorithm may be configured to generate a matrix based on the superpixels of two or more images. According to some embodiments, the image processing module 108 and/or the image processing algorithm may be configured to generate a cube based, at least in part, on the one or more of merged arrays. For example, the generated cube 400 may include the merged array 300. According to some embodiments, the cube (or generated cube) may include a 3D matrix.

According to some embodiments, the cube may include a first and second dimensions associated with spatial coordinates on the tongue of the subject. According to some embodiments, the first and second dimensions may include at least one of an x, y and/or z dimension of the spatial coordinates of the images and/or the tongue of the subject. According to some embodiments, the first and second dimensions may include a lateral coordinate system associated with the images and/or the tongue of the subject.

According to some embodiments, the cube may include an additional dimension associated with a 3D coordinate system associated with the images and/or the tongue of the subject.

According to some embodiments, the cube may include a third dimension associated with ranges of wavelengths of light corresponding to the spatial coordinates on the tongue of the subject. According to some embodiments, the cube may include a plurality of planes positioned along the third dimension. According to some embodiments, each plane of the plurality of planes may correspond with a specific a wavelength or range of wavelengths. According to some embodiments, at least a portion of the planes of the plurality of planes may be associated with different ranges of wavelengths. According to some embodiments, at least a portion of the planes of the plurality of planes may be associated with a specific spatial coordinate of the tongue of the subject. According to some embodiments, each pixel within one or more of the planes may include a value associated with the intensity of a wavelength or range of wavelengths of the plane. According to some embodiments, each plane may depict all or most of the spatial coordinates of the two or more images and/or the tongue of the subject. According to some embodiments, each plane may depict all or most of the spatial coordinates associated with one or more segments of the tongue of the subject.

According to some embodiments, the cube depicted in FIG. 4 shows a plurality of planes, such as, for example, planes 402a/402b/402c/402d (collectively referred to herein as planes 402) positioned along the third dimension depicted by arrow (Z). According to some embodiments, the individual pixels of the merged array 300 each correspond to a plane, such as pixel 1a corresponding to plane 402a, pixel 1b corresponding to plane 402b, and so on. According to some embodiments, the cube 400 may include a plurality of merged arrays, such as array 300, wherein pixels depicted specific ranges of wavelengths may be positioned in a same order in each of the merged arrays. According to some embodiments, each of the planes 402 includes a plurality of pixels from different merged arrays, wherein the pixels belonging to an individual plane may include only pixels of the same range of wavelengths.

According to some embodiments, the image processing module 108 may be configured to pre-process the images received from the image capturing module 106 and/or the cloud storage unit and/or the storage module 104. According to some embodiments, the image processing module 108 may be include one or more image processing algorithms. In some embodiments, the image processing module 108 may be configured to apply image processing algorithms to at least a portion of the one or more images. In some embodiments, the image processing module 108 may be configured to apply image processing algorithms to at least a portion of the generated cube. According to some embodiments, the image processing algorithms may include pre-processing and/or processing algorithms.

According to some embodiments, the pre-processing algorithms may be configured to perform any one or more of: image selection, image adjustment, accounting for motion blur, distortion, and/or data replication caused by motion of the tongue during the capturing of the image, normalization, noise reduction, color fidelity, texture enhancement, local contrast enhancement, local color contrast enhancement, geometric feature enhancement, image segmentation, image color segmentation, and motion detection. In some embodiments, each possibility is a separate embodiment.

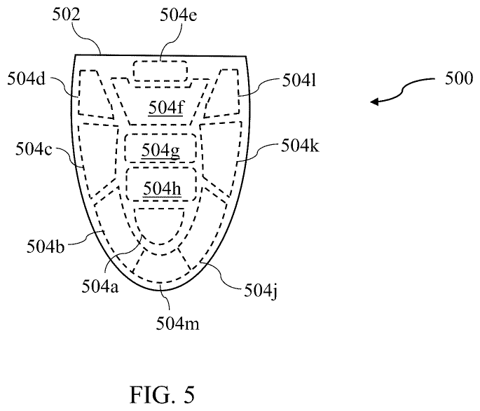

Reference is made to FIG. 5, which is a simplified illustration of a tongue with exemplary segmentations, in accordance with some embodiments of the present invention. According to some embodiments, the image segmentations may include a segmentation of the tongue, such as segmentation 502 of tongue 500. According to some embodiments, the image segmentation may include a plurality of sub-segmentations of the tongue, such as sub-segmentations 504a/504b/504c/504d/504e/504f/504g/504h/504i/504j/504k/504l/504m of tongue 500 (collectively referred to herein as sub-segments 504).

According to some embodiments, the image processing module 108 may include an algorithm configured to convert the one or more images obtained and/or captured by the image capturing module 106 to one or more RGB images. According to some embodiments, the image processing module 108 may implement one or more pre-processing and/or processing algorithms on the one or more RGB images, thereby obtaining a mask (such as, e.g., a segmentation mask). According to some embodiments, the image processing module may be configured to implement the pre-processing and/or processing algorithms on the one or more images (obtained and/or captured by the image capturing module 106) based, at least in part, on the sequences that were applied to the one or more RGB images.

According to some embodiments, the image processing module may be configured to implement the mask (or segmentation mask) on the one or more images and/or the cube. According to some embodiments, the image processing module may be configured to implement the pre-processing and/or processing algorithms on the generated cube based, at least in part, on the sequences that were applied to the one or more RGB images. For example, according to some embodiments, image segmentation, or an image segmentation algorithm associated with the pre-processing algorithm) may be implemented on the one or more RGB images. Once a segmentation and/or sub-segmentations are outlined onto the one or more RGB images, the segmentation coordinates can be used onto one or more images (obtained and/or captured by image capturing module 106).

According to some embodiments, the machine learning module 110 may be configured to generate a specific combination of operations for conversion of the cube, wherein the combination of operations may correspond to one or more disorders. According to some embodiments, different combinations of operations may correspond with different one or more disorders. According to some embodiments, the machine learning module 110 may be include a machine learning algorithm configured to generate a specific combination of operations for conversion of the cube, wherein the combination of operations may correspond to one or more disorders. According to some embodiments, the converted cube may include the product (or in other words, the result) of the combination of operations applied to the generated matrix. According to some embodiments, the one or more machine learning algorithms may include one or more deep learning methods. According to some embodiments, the one or more deep learning methods may utilize one or more neutral networks.

According to some embodiments, the machine learning algorithm may be configured to receive one or more images, the cube, and/or at least a portion of the cube. According to some embodiments, the machine learning algorithm may be configured to receive two or more images, the cube, and/or at least a portion of the cube. According to some embodiments, the machine learning algorithm may be configured to receive the cube, such as the cube 400.

According to some embodiments, the machine learning algorithm may be configured to output one or more gastrointestinal disorders associated with the tongue of the subject. According to some embodiments, the machine learning algorithm may be configured to output a combination of operations associated with one or more gastrointestinal disorders. According to some embodiments, the combination of operations may be configured to emphasize at least one mathematical relationship between two or more planes of the cube, wherein the at least one mathematical relationship may be associated with one or more gastrointestinal disorders. According to some embodiments, the combination of operations may be configured to enhance a contract between two or more planes of the cube, wherein the contrast may be associated with one or more gastrointestinal disorders. According to some embodiments, the combination of operations may be configured to differentiate between two or more planes of the cube. According to some embodiments, the combination of operations may be configured to combine two or more planes of the cube. According to some embodiments, the combination of operations may be configured to change the relative proportions of two or more planes of the cube. According to some embodiments, the combination of operations may be configured to reduce the number of total planes within the converted cube, for example, in relation to the generated cube.

According to some embodiments, the combination of operations may be configured to provide a specific weight, to one or more planes of the cube. According to some embodiments, the weight may include an index or coefficient. According to some embodiments, the weight may be linear or non-linear.

According to some embodiments, the combination of operations may include any one or more of addition, scalar multiplication, exponentiation, transposition, and transformation. According to some embodiments, the combination of operations may include at least one linear operation. According to some embodiments, the combination of operations may include at least one non-linear operation.

According to some embodiments, the combination of operations may include generating one or more submatrices. According to some embodiments, the one or more submatrices may be associated with one or more segmentations of the tongue of the subject. According to some embodiments, the combination of operations may include one or more operations applied at least to the one or more submatrices. According to some embodiments, the combination of operations may include one or more operations applied only to the one or more submatrices. According to some embodiments, the combination of operations may be applied onto one or more rows of the cube, such as, for example, one or more rows of pixels correlating with one or more merged arrays. For example, according to some embodiments, the combination of operations may be applied onto one or more individual merged arrays. According to some embodiments, the combination of operations may be applied to a group of rows within the cube, wherein the group of rows may be associated with a segmentation and/or sub-segmentation of the tongue of the subject. According to some embodiments, a row of the cube may include a merged array associated with a specific spatial coordinate on the tongue of the subject.

According to some embodiments, the combination of operations may include one or more operations that are applied to only a portion of the plurality of planes. According to some embodiments, the combination of operations may include one or more operations that are applied to any one or more of the planes within the cube which are normal to the third dimension, the planes within the cube which are normal to the second dimension, and/or the planes within the cube which are normal to the first dimension. According to some embodiments, the combination of operations may include one or more operations that are applied to one or more rows that may be parallel to any one of the first, second and/or third dimension. According to some embodiments, the combination of operations may include one or more operations that are applied to a selected group of rows and/or individual pixels within the cube.

According to some embodiments, the combination of operations may include generating one or more images. According to some embodiments, the combination of operations may include generating one or more images based, at least in part, on one or more planes of the cube. According to some embodiments, the combination of operations may include generating a plurality of images. According to some embodiments, the combination of operations may include generating a series of images. According to some embodiments, the combination of operations may include generating a scalar signal. According to some embodiments, the combination of operations may include generating a plurality of scalar signals. According to some embodiments, the combination of operations may include applying a one-dimensional and/or two-dimensional function to the cube or at least a portion of the cube. According to some embodiments, the machine learning algorithm may be trained using a training set including a plurality of matrices corresponding to a plurality of tongues of a plurality of subjects. According to some embodiments, the machine learning algorithm may be trained using a training set including a plurality of labels associated with the plurality of cubes. According to some embodiments, the plurality of labels may include indications of at least one gastrointestinal disorder associated with the corresponding plurality of subjects.

According to some embodiments, the one or more machine learning modules 110 may include a machine learning algorithm configured to classify output data associated with the generated cube as being associated with one or more gastrointestinal disorders. According to some embodiments, the machine learning algorithm may include one or more classifying algorithms. According to some embodiments, the machine learning algorithm may include one or more deep learning algorithms including a plurality of neural networks.

According to some embodiments, the machine learning algorithm may be configured to receive and/or generate the cube based on the superpixels and/or the merged arrays associated with the one or more images. According to some embodiments, the machine learning algorithm may be configured to receive a specific combination of operations corresponding to one or more gastrointestinal disorders, such as the combination of operations as described hereinabove. According to some embodiments, the machine learning algorithm may be configured to receive the specific combination of operations from any one or more of the processor 102, the memory module 104, and from a different algorithm within the one or more machine learning modules 110. According to some embodiments, the machine learning algorithm may be configured to receive the cube as input. According to some embodiments, the machine learning algorithm may be configured to classify the cube as being associated with one or more disorders. According to some embodiments, the machine learning algorithm may be configured to classify the cube as being associated with one or more disorders without operations and/or mathematical manipulations of the cube. According to some embodiments, the machine learning algorithm may be configured to output one or more disorders associated with the received cube.

According to some embodiments, the machine learning algorithm may be configured to convert the cube using the specific combination of operations. According to some embodiments, the converted cube may include output data after applying the combination of operations to the received and/or generated cube. According to some embodiments, the machine learning algorithm may be configured to convert the cube into output data. According to some embodiments, the output data may include a single multispectral image in the form of a cube. According to some embodiments, the single multispectral image may be based, at least in part, on received and/or generated cube. According to some embodiments, the single image may be based, at least in part, on the converted cube. According to some embodiments, the single multispectral image may be a hyperspectral image.

According to some embodiments, the output data may include one or more submatrices. According to some embodiments, the one or more submatrices may be associated with one or more segmentations of the tongue of the subject. According to some embodiments, the output data may include a plurality of images. According to some embodiments, the output data may include a series of images. According to some embodiments, the output data may include a scalar signal. According to some embodiments, the output data may include a plurality of scalar signals. According to some embodiments, the output data may include a one-dimensional and/or two-dimensional function of the cube or at least a portion of the cube.

According to some embodiments, the machine learning algorithm may be configured to classify the output data as being associated with one or more disorders, based, at least in part, on the one or more ranges of wavelengths depicted within the output data. According to some embodiments, the machine learning algorithm may be configured to classify the output data as being associated with one or more disorders, based, at least in part, on the values of the intensities associated with one or more spatial coordinates within one or more planes depicted within the output data. According to some embodiments, the machine learning algorithm may be configured to classify the output data as being associated with one or more disorders, based, at least in part, on the amplitude—of the wavelengths of the one or more ranges of wavelengths depicted within the output data.

According to some embodiments, the machine learning algorithm may be configured to evaluate one or more threshold values for the amplitudes of the wavelengths of the one or more ranges of wavelengths depicted within the output data, wherein the threshold value may be associated with a gastrointestinal disorder. According to some embodiments, the machine learning algorithm may be configured to classify the output data as being associated with one or more disorders, based, at least in part, on the threshold values of the amplitudes. According to some embodiments, the machine learning algorithm may be configured to classify the output data as being associated with one or more disorders, based, at least in part, on the proportions or relative weights of two or more planes within the cube. According to some embodiments, the machine learning algorithm may be configured to classify the output data and/or the cube as being associated with one or more disorders, based, at least in part, on the values (or intensities) of one or more pixels within one or more planes associated with a specific wavelengths or ranges of wavelengths.

According to some embodiments, the machine learning algorithm may be configured to output the classified disorder associated with the one or more images of the tongue of the subject.

According to some embodiments, the system 100 may include a user interface module. According to some embodiments, the user interface module may be configured to receive data from a user or operator, such as, for example, age, gender, blood pressure, eating habits, risk factors associated with specific disorders, genetic data, medical history of the family of the subject and medical history of the subject. According to some embodiments, the user interface module may be configured to communicate with the processor 102 such that the user inputted data is fed to the one or more machine learning modules 110. According to some embodiments, the user interface module may include at least one of a display screen and a button. According to some embodiments, the user interface module may include a software configured for transferring inputted information from a user to the processor 102. According to some embodiments, the user interface module may include a computer program and/or a smartphone application. According to some embodiments, the user interface module may include a keyboard. According to some embodiments, the user interface module may be configured to receive data from the processor 102 and/or display data received from the processor 102. According to some embodiments, the user interface module may be configured to display a result of a detection of a disorder. According to some embodiments, the user interface module may be configured to display one or more outputs of the one or more machine learning modules 110.

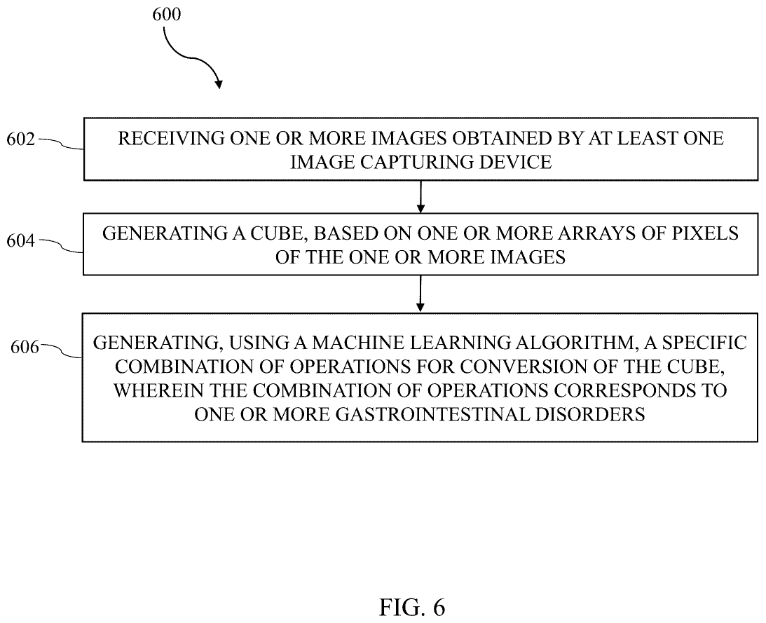

Reference is made to FIG. 6, which is a flowchart of functional steps in a process for detection of gastrointestinal disorders, in accordance with some embodiments of the present invention.

According to some embodiments, at step 602, method 600 may include receiving the one or more images obtained by the at least one image capturing device. According to some embodiments, at step 604, the method 600 may include generating a cube, based on one or more arrays of pixels of the one or more images. According to some embodiments, at step 606, the method 600 may include generating, using a machine learning algorithm, a specific combination of operations for conversion of the cube, wherein the combination of operations corresponds to one or more gastrointestinal disorders.

According to some embodiments, the method 600 may include obtaining a plurality of images of a tongue of a subject. According to some embodiments, at step 602, method 600 may include receiving the one or more images obtained by the at least one image capturing device. According to some embodiments, the one or more images may be multispectral images. According to some embodiments, the one or more images may be hyperspectral images. According to some embodiments, the one or more images may include a combination of multispectral and hyperspectral images. According to some embodiments, each of the one or more images may include superpixels, each depicting a specified range of wavelengths of light reflected from the tongue of the subject.

According to some embodiments, the method may include applying image pre-processing algorithms to the one or more images. According to some embodiments, the method may include applying image processing algorithms to the one or more images. According to some embodiments, the method may include aligning and/or merging arrays based on two or more superpixels which correspond to a same spatial coordinate on the tongue of the subject. According to some embodiments, the method may include generating a merged array based, at least in part, on two or more superpixels of two or more images, wherein the two or more superpixels are associated with a same spatial coordinate on the tongue of the subject. According to some embodiments, the method may include arranging the pixels within the merged array. According to some embodiments, the method may include identifying two or more replica pixels which may be associated with a same range of wavelengths and a same spatial coordinate on the tongue of the subject. According to some embodiments, the method may include removing at least one of the two or more replica pixels.

According to some embodiments, at step 604, the method 600 may include generating a cube, based on two or more superpixels of the one or more images.

According to some embodiments, the cube may include at least a first and second dimensions associated with spatial coordinates on the tongue of the subject. According to some embodiments, the cube may include at least a third dimension associated with ranges of wavelengths of light corresponding to the spatial coordinates on the tongue of the subject.

According to some embodiments, the method may include applying image pre-processing algorithms and/or image processing to the cube. According to some embodiments, the method may include applying image pre-processing algorithms and/or image processing to at least a portion of the cube. According to some embodiments, the method may include performing any one or more of image selection, image adjustment, accounting for motion blur, distortion, and/or data replication caused by motion of the tongue during the capturing of the image, normalization, noise reduction, color fidelity, texture enhancement, local contrast enhancement, local color contrast enhancement, geometric feature enhancement, image segmentation, sub-segmentation, image color segmentation, and motion detection.

According to some embodiments, the method may include converting the one or more images to one or more RGB images. According to some embodiments, the method may include applying one or more pre-processing and/or processing algorithms on the one or more RGB images, thereby generating a mask, such as, for example, a segmentation mask. According to some embodiments, the method may include segmenting the one or more RGB images into segments of the tongue, such as, for example, as depicted in FIG. 5. According to some embodiments, the method may include recording the sequences of pre-processing and/or image processing that were applied to the one or more RGB images. According to some embodiments, the method may include applying the mask (or segmentation mask) to the captured images and/or to the cube. According to some embodiments, the method may include applying sequences of pre-processing and/or image processing to the cube, based, at least in part, on the sequences of pre-processing and/or image processing that were applied to the one or more RGB images.

According to some embodiments, at step 606, the method 600 may include generating, using a machine learning algorithm, a specific combination of operations for conversion of the cube, wherein the combination of operations corresponds to one or more gastrointestinal disorders, such as described hereinabove. According to some embodiments, the method may include outputting a combination of operations associated with one or more gastrointestinal disorders. According to some embodiments, the method may include outputting a combination of operations for different individual gastrointestinal disorder.

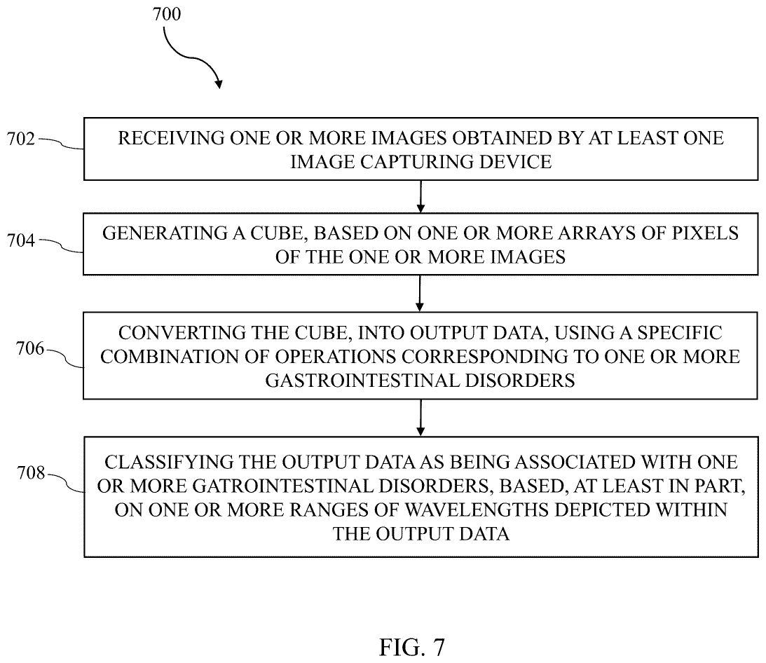

Reference is made to FIG. 7, which is a flowchart of functional steps in a process for detection of gastrointestinal disorders, in accordance with some embodiments of the present invention. According to some embodiments, method 700 may be a continuation of method 600. According to some embodiments, the method 700 may include at least a portion of method 600. According to some embodiments, method 700 may include some or all of the steps of method 600.

According to some embodiments, at step 702, the method 700 may include receiving the one or more images obtained by the at least one image capturing device. According to some embodiments, at step 704, the method 700 may include generating a cube, based on one or more arrays of pixels of the one or more images. According to some embodiments, at step 706, the method 700 may include converting the cube, into output data, using a specific combination of operations corresponding to one or more gastrointestinal disorders. According to some embodiments, at step 708, the method 700 may include classifying the output data as being associated with one or more gastrointestinal disorders, based, at least in part, on the one or more ranges of wavelengths depicted by the output data.

According to some embodiments, the method 700 may include obtaining one or more images of a tongue of a subject. According to some embodiments, the method 700 may include obtaining a plurality of images of a tongue of a subject. According to some embodiments, at step 702, method 700 may include receiving the one or more images obtained by the at least one image capturing device. According to some embodiments, the one or more images may be multispectral images. According to some embodiments, the one or more images may be hyperspectral images. According to some embodiments, the one or more images may include a combination of multispectral and hyperspectral images. According to some embodiments, each of the one or more images may include superpixels, each depicting a specified range of wavelengths of light reflected from the tongue of the subject.

According to some embodiments, the method may include applying image pre-processing algorithms to the one or more images. According to some embodiments, the method may include applying image processing algorithms to the one or more images. According to some embodiments, the method may include generate one or more arrays based, at least in part, on one or more superpixels of the one or more images. According to some embodiments, the method may include aligning and/or merging two or more arrays based on two or more superpixels, wherein the two or more superpixels correspond to a same spatial coordinate on the tongue of the subject. According to some embodiments, the method may include generating a merged array based, at least in part, on two or more superpixels of two or more images, wherein the two or more superpixels are associated with a same spatial coordinate on the tongue of the subject. According to some embodiments, the method may include arranging the pixels within the merged array. According to some embodiments, the method may include identifying two or more replica pixels which may be associated with a same range of wavelengths and a same spatial coordinate on the tongue of the subject. According to some embodiments, the method may include removing at least one of the two or more replica pixels.

According to some embodiments, at step 704, the method 700 may include generating a cube, based on one or more arrays of pixels of the one or more images.

According to some embodiments, the cube may include at least a first and second dimensions associated with spatial coordinates on the tongue of the subject. According to some embodiments, the cube may include at least a third dimension associated with ranges of wavelengths of light corresponding to the spatial coordinates on the tongue of the subject.

According to some embodiments, the method may include applying image pre-processing algorithms and/or image processing to the cube. According to some embodiments, the method may include applying image pre-processing algorithms and/or image processing to at least a portion of the cube. According to some embodiments, the method may include performing any one or more of image selection, image adjustment, accounting for motion blur, distortion, and/or data replication caused by motion of the tongue during the capturing of the image, normalization, noise reduction, color fidelity, texture enhancement, local contrast enhancement, local color contrast enhancement, geometric feature enhancement, image segmentation, sub-segmentation, image color segmentation, and motion detection.

According to some embodiments, the method may include converting the one or more images to one or more RGB images. According to some embodiments, the method may include applying one or more pre-processing and/or processing algorithms on the one or more RGB images, thereby generating a mask, such as a segmentation mask. According to some embodiments, the method may include segmenting the one or more RGB images into segments of the tongue, such as, for example, as depicted in FIG. 5. According to some embodiments, the method may include recording the sequences of pre-processing and/or image processing that were applied to the one or more RGB images. According to some embodiments, the method may include applying sequences of pre-processing and/or image processing to the cube, based, at least in part, on the sequences of pre-processing and/or image processing that were applied to the one or more RGB images. According to some embodiments, the method may include applying the mask (or segmentation mask) to the cube. According to some embodiments, the method may include receiving a specific combination of operations for conversion of the cube, wherein the combination of operations corresponds to one or more gastrointestinal disorders, such as described hereinabove. According to some embodiments, the method may include obtaining the combination of operations for conversion of the cube using one or more machine learning algorithms, such as, for example, the one or more machine learning modules 110 and/or as depicted in method 600. According to some embodiments, the method may include obtaining the combination of operations for conversion of the cube from the storage module, such as storage module 104.

According to some embodiments, at step 706, the method 700 may include converting the cube, into data output, using a specific combination of operations corresponding to one or more gastrointestinal disorders.

According to some embodiments, the method may include generating output including one or more submatrices. According to some embodiments, the method may include generating output including a plurality of images. According to some embodiments, the method may include generating output including a series of images. According to some embodiments, the method may include generating output including a scalar signal. According to some embodiments, the method may include generating output including a plurality of scalar signals.

According to some embodiments, the generating the output data may include applying one or more analyses to the cube. According to some embodiments, generating the output data may include applying one or more principal component analysis techniques. According to some embodiments, generating the output may include applying one or more principal component regression techniques.

According to some embodiments, the method may include classifying the cube as being associated with one or more gastrointestinal disorders, based, at least in part, on the one or more ranges of wavelengths depicted within the pixel of the cube. According to some embodiments, the method may include classifying the output data as being associated with one or more gastrointestinal disorders, based, at least in part, on the one or more ranges of wavelengths depicted by the output data. According to some embodiments, the method may include classifying the output data as being associated with one or more gastrointestinal disorders, based, at least in part, on the values of the intensities associated with one or more spatial coordinates within one or more planes depicted by the output data. According to some embodiments, the method may include classifying the output data as being associated with one or more gastrointestinal disorders, based, at least in part, on the amplitude of the wavelengths of the one or more ranges of wavelengths depicted by the output data. According to some embodiments, the method may include evaluating one or more threshold values for the amplitudes of the wavelengths of the one or more ranges of wavelengths depicted within the output data, wherein the threshold value may be associated with a gastrointestinal disorder. According to some embodiments, the method may include classifying the output data as being associated with one or more disorders, based, at least in part, on the threshold values of the amplitudes.

According to some embodiments, the method may include classifying the output data as being associated with one or more gastrointestinal disorders, based, at least in part, on the proportions or relative weights of two or more planes depicted by the cube. According to some embodiments, the method may include classifying the output data and/or the cube as being associated with one or more gastrointestinal disorders, based, at least in part, on the values (or intensities) of one or more pixels within one or more planes associated with a specific wavelengths or ranges of wavelengths.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. A system for detecting gastrointestinal disorders utilizing one or more multispectral images of a tongue of a subject, comprising:
at least one hardware processor in communication with the at least one image capturing device configured to capture at least one multispectral image of a tongue of the subject in real time; and
a non-transitory computer-readable storage medium having stored thereon program code, the program code executable by the at least one hardware processor to:
receive the at least one multispectral image obtained by the at least one image capturing device, wherein the at least one multispectral image comprises at least one superpixel associated with spatial coordinates on the tongue of the subject, each pixel of the at least one superpixel depicting a specified range of wavelengths of light;
generate a cube, based on the superpixel, of the at least one multispectral images, the cube comprising at least:
a first and second dimensions associated with spatial coordinates on the tongue of the subject, and
a third dimension associated with ranges of wavelengths of light corresponding to the spatial coordinates on the tongue of the subject;
generate, using a machine learning algorithm, a specific combination of operations for conversion of the cube, and wherein the combination of operations corresponds to one or more gastrointestinal disorders.

2. The system according to claim 1, wherein the at least one superpixels is in the form of a 3 by 3 or 4 by 4 or 5 by 5 or 6 by 6 matrix.

3. The system according to claim 1, wherein the combination of operations is configured to emphasize at least one mathematical relationship between two or more planes of the cube associated with one or more gastrointestinal disorders.

4. The system according to claim 1, wherein the cube comprises a plurality of planes along the third dimension, each plane corresponding to a wavelength or range of wavelengths wherein the combination of operations is configured to provide a specific weight to different planes of the cube and/or wherein the combination of operations comprises at least one specific operation that is applied to only a portion of the plurality of planes.

5. (canceled)

6. (canceled)

7. (canceled)

8. The system according to claim 1, wherein the combination of operations comprises at least one non-linear operation.

9. The system according to claim 1, wherein the combination of operations is configured to reduce the number of total planes of the converted matrix in relation to the cube.

10. The system according to claim 1, wherein the machine learning algorithm is trained using a training set comprising:
a plurality of cubes corresponding to a plurality of tongues of a plurality of subjects, and
a plurality of labels associated with the plurality of cubes, each label indicating at least one medical disorder associated with the corresponding plurality of subjects,
wherein the at least one hardware processor is in communication with at least one image capturing device, wherein the at least one image capturing device is configured to capture at least one multispectral image of a tongue of a subject in real time.

11. (canceled)

12. A system for detecting gastrointestinal disorders utilizing one or more multispectral images of a tongue of a subject, comprising:
at least one hardware processor in communication with the at least one image capturing device configured to capture at least one multispectral image of a tongue of the subject in real time; and
a non-transitory computer-readable storage medium having stored thereon program code, the program code executable by the at least one hardware processor to:
receive the at least one multispectral image obtained by the at least one image capturing device, wherein the at least one multispectral image comprises at least one superpixel associated with spatial coordinates on the tongue of the subject, each pixel of the at least one superpixel depicting a specified range of wavelengths of light;
generate a cube, based on the at least one superpixel of the at least one multispectral image, the cube comprising at least:
a first and second dimensions associated with spatial coordinates on the tongue of the subject, and
a third dimension associated with ranges of wavelengths of light corresponding to the spatial coordinates on the tongue of the subject;
convert the cube, into output data, using a specific combination of operations corresponding to one or more gastrointestinal disorders; and
classify the output data as being associated with one or more gastrointestinal disorders, based, at least in part, on one or more ranges of wavelengths depicted within the output data.

13. (canceled)

14. (canceled)

15. (canceled)

16. (canceled)

17. The system according to claim 12, wherein the program code is further executable to classify the single multispectral image as being associated with one or more gastrointestinal disorders, based, at least in part, on a machine learning algorithm configured to receive the converted cube and output one or more gastrointestinal disorders corresponding to any one or more of the values of one or more pixels, proportions or relative weights of the planes, amplitudes of specific wavelengths or ranges of wavelengths and intensities of specific wavelengths or ranges of wavelengths, of the converted cube.

18. (canceled)

19. (canceled)

20. The system according to claim 12, wherein the at least one hardware processor is in communication with at least one image capturing device, wherein the at least one image capturing device is configured to capture at least one multispectral image of a tongue of a subject in real time.

21. (canceled)

22. The system according to claim 20, wherein the at least one image capturing device comprises at least two cameras and wherein the at least two cameras are positioned in an optical path of a beamsplitter such that each of the at least two cameras obtain a separate spectrum of light reflected from the tongue.

23. (canceled)

24. The system according to claim 20, wherein the at least one image capturing device comprise at least two sensors, wherein each sensor comprises a plurality of lenses each configured to capture a wavelength or range of wavelengths.

25. (canceled)

26. (canceled)

27. (canceled)

28. (canceled)

29. The system according to claim 12, wherein the at least one hardware processor is in communication with at least two image capturing devices and wherein the ranges of wavelengths obtained by the at least two image capturing devices are different.

30. (canceled)

31. (canceled)

32. The system according to claim 12, wherein at least one of the ranges of wavelengths range within 470 and 900 nm.

33. The system according to claim 12, wherein the at least one multispectral image comprises a video segment.

34. (canceled)

35. (canceled)

36. (canceled)

37. The system according to claim 12, wherein a maximal exposure time of the at least one image captured by the at least one image capturing device is about 100 msec.

38. The system according to claim 12, wherein the multispectral image comprises over about 25 active bands.

39. (canceled)

40. (canceled)

41. A method for detecting gastrointestinal disorders utilizing one or more multispectral images of a tongue of a subject, the method comprising:
obtaining a plurality of multispectral images of a tongue of the subject wherein each image includes at least one superpixels, depicting a specified range of wavelengths of light reflected from the tongue of the subject;
merging the plurality of multispectral images, thereby forming a cube comprising at least:
a first and second dimensions associated with spatial coordinates on the tongue of the subject, and
a third dimension associated with ranges of wavelengths of light corresponding to the spatial coordinates on the tongue of the subject;
converting the cube, into output data, based, at least in part, on a specific combination of operations corresponding to one or more gastrointestinal disorders; and
classifying the output data as being associated with one or more gastrointestinal disorders, based, at least in part, on the one or more ranges of wavelengths depicted by the output data.

42. (canceled)

43. (canceled)

44. (canceled)

45. The method according claim 41, wherein the cube comprises a plurality of planes along the third dimension, each plane corresponding to a wavelength or range of wavelengths.

46. (canceled)

47. (canceled)

48. (canceled)

49. (canceled)

50. The method according to claim 41, wherein the ranges of wavelengths are different.

51. (canceled)

52. (canceled)

53. (canceled)

54. (canceled)

55. (canceled)

56. (canceled)

57. (canceled)
